(19) United States
(12) Patent Application Publication
(10) Pub. No.: US 2012/0041277 A1
(43) Pub. Date: Feb. 16, 2012

(54) SYSTEM AND METHOD FOR PREDICTING NEAR-TERM PATIENT TRAJECTORIES

(75) Inventors: SHAHRAM EBADOLLAHI, Tarrytown, NY (US); Jianying Hu, Bronx, NY (US); Robert K. Sorrentino, Rancho Palos Verdes, CA (US); Daby M. Sow, Croton On Hudson, NY (US); Jimeng Sun, White Plains, NY (US)

(73) Assignee: INTERNATIONAL BUSINESS MACHINES CORPORATION, Armonk, NY (US)

(21) Appl. No.: 12/855,068

(22) Filed: Aug. 12, 2010

(51) Int. Cl. A61B 5/00

(52) U.S. Cl. 600/301

(57) ABSTRACT

A system and method for predicting near term measurements of a patient includes a stream processor configured to summarize raw measurements from patients into signatures and construct optimal prediction models based on previously obtained signatures. A similar patient tracker is configured to monitor similar patient information for a query patient. The similar patient information is determined based on a similarity between the query patient and signatures of other patients. A model analyzer is configured to employ retrofitted optimal prediction models from similar patients to predict near term measurements of the query patient.

### CROSS-REFERENCE TO RELATED APPLICATIONS

This application is related to commonly assigned U.S. application Ser. No. [TBD], entitled “SYSTEM AND METHOD FOR PREDICTING LONG-TERM PATIENT OUTCOME”, Attorney Docket Number YOR920100439US1(163-357), filed concurrently herewith, which is incorporated by reference herein in its entirety.

### BACKGROUND

1. Technical Field

The present invention relates to predicting of near term prognoses of patients and more particularly to systems and methods for near term prognosis using information about similar patients.

2. Description of the Related Art

The task of prognosis is an important component of the process of clinical care. It is about predicting the future health status of the patient and the probable course of her health indicators. Oftentimes, clinicians are concerned about the near term potential trajectory of a number of Key Patient Indicators (KPIs) for a given patient such as echo cardiogram (ECG), blood pressure, heart rate, SpO₂. Accurately, predicting near term trajectories is needed to improve clinical effectiveness and efficiency. Usually, the prediction of trajectories of a patient is performed based on the data from that same patient, and predefined rules based on prior knowledge of clinicians and expert guidelines.

### SUMMARY

A system and method for predicting near term measurements of a patient include a stream processor configured to summarize raw measurements from patients into signatures and construct optimal prediction models based on previously obtained signatures. A similar patient tracker is configured to monitor similar patient information for a query patient. The similar patient information is determined based on a similarity between the query patient and signatures of other patients. A model analyzer is configured to employ retrofitted optimal prediction models from similar patients to predict near term measurements of the query patient.

A method for predicting near term measurements of a patient includes summarizing time windows of patient measurements into signatures; retrofitting optimal prediction models based on historical measurements; tracking similar patients and signatures of the similar patients for a query patient; constructing a predictive model of the query patient using the optimal models from the similar patients; and predicting a near-term measurement for the query patient based on the predictive model.

These and other features and advantages will become apparent from the following detailed description of illustrative embodiments thereof, which is to be read in connection with the accompanying drawings.

### BRIEF DESCRIPTION OF DRAWINGS

The disclosure will provide details in the following description of preferred embodiments with reference to the following figures wherein:

FIG. 1 is a block/flow diagram showing a system/method for retrieving patients with similarities for near term patient prognosis based on similar patients in accordance with one illustrative embodiment;

FIG. 2 is a block/flow diagram showing a system/method for near term patient prognosis in accordance with one illustrative embodiment;

FIG. 3 is a block/flow diagram showing a system/method for computing a patient signature in accordance with one illustrative embodiment;

FIG. 4 is a block/flow diagram showing a system/method for retrofitting models to form a prediction model in accordance with one illustrative embodiment; and

FIG. 5 is a block/flow diagram showing a system/method for near term patient prognosis based on similar patients in accordance with another illustrative embodiment.

### DETAILED DESCRIPTION OF PREFERRED EMBODIMENTS

In accordance with the present principles, systems and methods for predicting near term prognosis of patients are provided. Current systems do not predict near term trajectories for a query patient using historical data or patterns from similar patients. Historical data from similar patients can help provide better estimate future events for the query patient, and can help determine different treatment options and their expected outcome. The method and infrastructure reliably and efficiently monitor and extract similar patient information in near real time.

In one embodiment, a compact summarization of longitudinal data streams of patient Key Patient Indicators (KPIs) is provided. An efficient similar patient matching method leverages compact summarization of patient KPIs, and a predictive model is updated and provides forecasts based on similar patients.

A stream processor summarizes the multiple longitudinal patient data streams into a small set of signature representations, and a similar patient tracker incrementally maintains the set of similar patients and reference timestamps. A predictive model leverages all the model parameters from similar patients to build a model for predicting the query patient.

Effective patient similarity assessment may be employed for clinical decision support. This similarity assessment enables the capture of past experience as manifested in the collective longitudinal medical records of patients and helps clinicians assess the likely outcomes resulting from their decisions and actions. A patient similarity metric is one that is clinically relevant and semantically sound. Patient similarity is highly context sensitive: it depends on factors such as the disease, the particular stage of the disease, and co-morbidities. One way to discern the semantics in a particular context is to take advantage of physicians' expert knowledge as reflected in labels assigned to some patients. A method that leverages localized supervised metric learning to effectively incorporate such expert knowledge to arrive at semantically sound patient similarity measures is disclosed.

Medical records capture both observations of patients' health status, and decisions and actions taken by clinicians and care providers. Buried inside these records are insights on temporal evolution patterns of patient health status, and the effects of different clinical decisions on the trajectory of a disease. Tapping into this source of insight can be achieved by developing techniques measuring cross patient similarities. These techniques can improve patients' clinical outcomes as tools for diagnostic and prognostic decision support.

As will be appreciated by one skilled in the art, aspects of the present invention may be embodied as a system, method or computer program product. Accordingly, aspects of the present invention may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, aspects of the present invention may take the form of a computer program product embodied in one or more computer readable medium(s) having computer readable program code embodied thereon.

Any combination of one or more computer readable medium(s) may be utilized. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium may be transmitted using any appropriate medium, including but not limited to wireless, wireline, optical fiber cable, RF, etc., or any suitable combination of the foregoing. Computer program code for carrying out operations for aspects of the present invention may be written in any combination of one or more programming languages, including an object oriented programming language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Aspects of the present invention are described below with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks. The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

The flowchart and block diagrams in the Figures illustrate the architecture, functionality, and operation of possible implementations of systems, methods and computer program products according to various embodiments of the present invention. In this regard, each block in the flowchart or block diagrams may represent a module, segment, or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

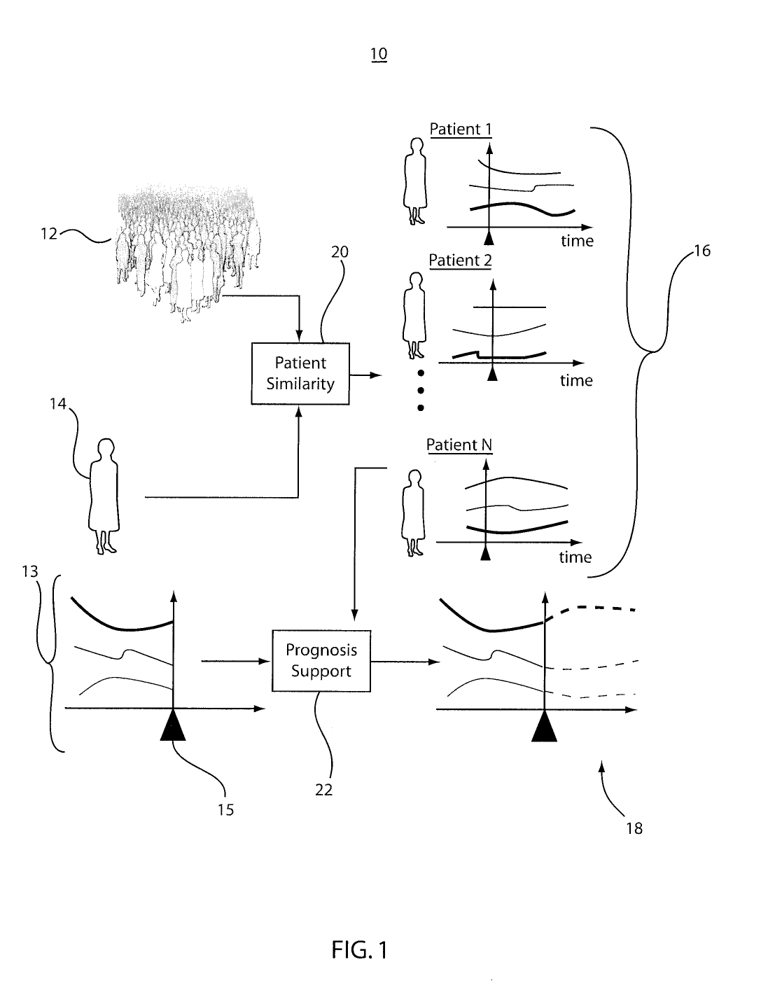

Referring now to the drawings in which like numerals represent the same or similar elements and initially to FIG. 1, a diagram illustratively shows a system 10 where a patient 14 with available observations 13 comes to a decision point 15. Patients 12 who are clinically similar to a query patient 14 are retrieved. The system 10 is provided with decisions and actions 16 for a plurality of similar patients (patient 1, patient 2, etc.) and their consequences and decides on a best course of action for the current patient 14. In addition, the system 10 can project a trajectory 18 of patient's health status, as captured by the patient's clinical factors and biomarkers, under the regime of any particular decision made.

The system 10 provides an alignment of the trajectories of patients' temporal characteristics to make the records amenable to semantically and clinically sound comparison. The system 10 devises similarity measures 20 that can reflect the clinical proximity or disparity between different patients. The system 10 couples decisions and their consequences using patient prognosis support 22.

Different methods for feature generation over multi-dimensional temporal patient data may be employed. In one embodiment, a localized supervised metric learning approach can be adopted to arrive at a semantically sound similarity measure for retrieving patients represented in the multi-dimensional feature space.

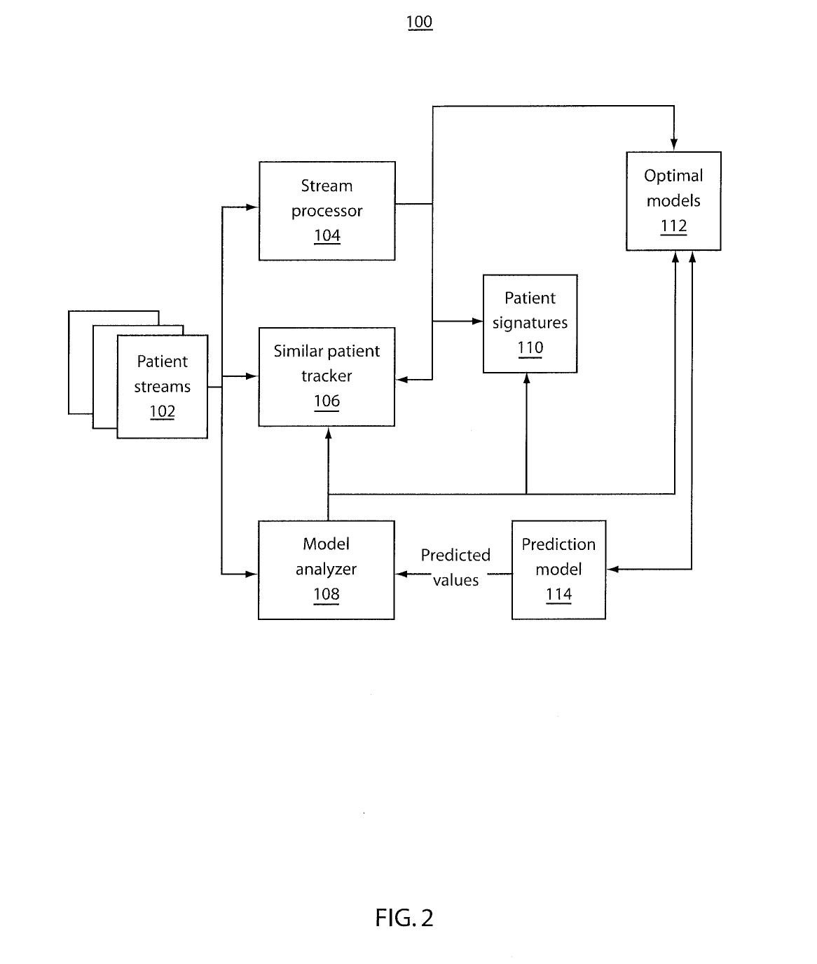

Referring to FIG. 2, a system 100 provides for predictions of near term prognosis of patients. System 100 may include on a single processing device such as a computer, a personal digital assistant or other computing device, or may include a plurality of distributed computers in a network environment or the like. Patient data streams 102 are input to the system 100. The input data streams 102 provide patient vital signs, such as, e.g., Key Patient Indicators (KPIs) or other relevant patient information. A stream processor 104 performs a compact summarization of longitudinal data streams of the patient KPIs. The stream processor 104 summarizes the multiple longitudinal patient data streams into a small set of signature representations stored in a patient signature database 110. The signatures may include a multidimensional vector that includes relevant information for that patient for a particular time window. For example, features may include heart rate, blood oxygen, temperature, etc.

A similar patient tracker 106 performs an efficient similar patient matching method that leverages the compact summarization of patient KPIs. The similar patient tracker 106 incrementally maintains the set of similar patients and a reference timestamp.

Updates and forecasting based on similar patients is performed using a model analyzer 108. A predictive model 114 leverages all the model parameters from similar patients stored in optimal model storage 112 to build the model for predicting a query patient. Models may take many forms, but may be constructed using the relevant medical factors in the particular instance, e.g., for that disease, for that treatment program, etc.

The stream processor 104, in real-time, summarizes raw input streams 102 such as ECG, blood pressure, SpO2 measures to extract representative signatures and store them in storage 110. Stream processor 104 retrospectively constructs/updates the optimal predictive model 114 to predict the current measurements based on historical measurements. The similar patient tracker 106 updates the top similar patients (e.g., k patients) to each patient x based on the recent measurements from x. The similar patient tracker 106 constructs current signature based current measurements, and then searches the database 110 of existing patient signatures to find matching signatures of those existing patients. For each patient x, the similar patient tracker 106 can choose to maintain either a single set of similar patients to x, or different sets of similar patients for each type of measurement. For example, system 100 tracks two types of measurements ECG and SpO2. For patient x, the similar patient tracker 106 can choose to maintain either an overall set of similar patients to x, or two sets of similar patients, one set based on ECG and one set based on SpO2.

The prediction model 114 may be refreshed periodically or may be refreshed in accordance with data characteristics. For example, as the SpO₂ reading changes, the model may be updated. The model analyzer 108 finds the optimal models 112 of the top “k” similar patients to each patient x, and then constructs a forecasting model or prediction model 114 based on the optimal models 112 and predicts the future measurement of x. A prediction error will be fed back to the similar patient tracker 106 to determine/update the top-k similar patients for x.

The patient signature database 110 includes patient signatures. A patient signature is a low-dimensional representation of the patient condition in a certain time window. Each patient can have multiple signatures based on the variation of his/her characteristics. Signature construction includes a time series of measurements as input, and a small set of signatures (low dimensional signatures) are output.

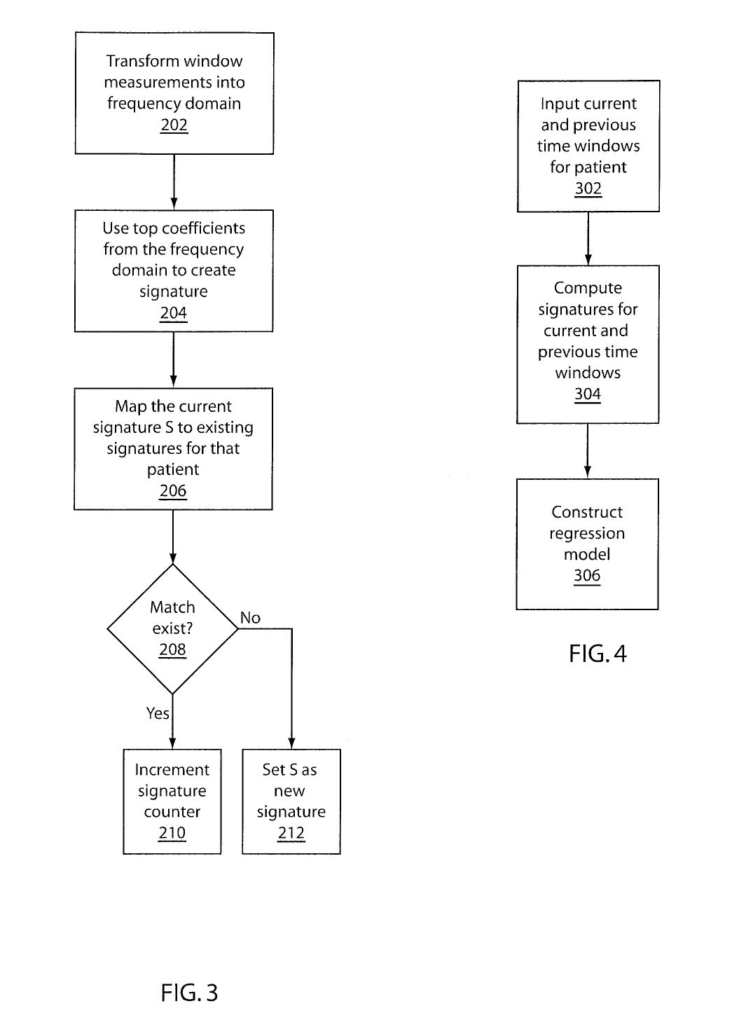

Referring to FIG. 3, an illustrative method is described for generating a new patient signature in accordance with an exemplary embodiment. In block 202, for each time window, W, over a time series, transform W into the frequency domain, e.g., using Fast Fourier Transformation (FFT) or Wavelets. In block 204, use the top coefficients from the frequency domain as a current signature S. In block 206, map S to existing signatures of this patient. In block 208, determine if a match exits. In particular, we mark two signatures matched if the Euclidean distance of two signature vectors is small (e.g., within a threshold). In block 210, if a match exists, then increment a counter of that signature by 1. Otherwise, in block 212, insert S as a new signature.

A signature search is one method for tracking and finding similar patients. Since each signature may include a low-dimensional vector, a tree structure may be employed to index all the signatures to speed up any search.

Referring to FIG. 4, the models are retrofitted to provide optimal models 112. The optimal model retrofitting includes inputting for each patient x, a current time window Wₜ and previous time windows Wₜ₋₁ in block 302. In block 304, signatures Sₜ and Sₜ₋₁ are computed for current and previous windows for each patient x. In block 306, a regression model of Sₜ=f(Sₜ₋₁) with parameter vector b, is constructed. Note that bₓ is the model parameters of the regression model. The regression model may include any statistical function. In particular, the regression model may be employed in the prediction model for similar patients.

Referring to again to FIG. 2, the prediction model 114 uses models from similar patients. The prediction model 114 receives as input, information on the query patient x and a previous time window Wₜ₋₁ for query patient x. In block 110, signatures for the query patient are computed. The similar patient tracker 106 employs the signatures to extract similar patients and their retrofitting model parameters (from optimal models 112). An ensemble model or prediction model 114 is constructed from the retrofitted models of similar patients. For example, weighted averages may be computed for all the retrofitting models, where the weight of each patient may be proportional to a similarity score. The prediction model 114 is employed to predict near-term outcomes for the query patient based upon similar patients.

Patient similarity measures may be determined in a plurality of ways. In one particularly useful embodiment, similarity measures may be determined using localized supervised metric learning (LSML) to provide a patient similarity measure. When a physician looks for similar patients in a database, the similarity is often based not only on quantitative measurements such as lab results, sensor measurements, age and sex, but also on the physician's assessment of the disease type and stage. The assessment would potentially influence the relative importance a physician places on different measurements or groups of measurements. To compute this specific notion of similarity, a distance metric is needed that can automatically adjust the importance of each numeric feature by leveraging the physician's belief.

Formally, quantitative measurements of a patient are represented by an N-dimensional feature vector x. Examples of features are the mean and variance of the sensor measures, or Wavelet coefficients. The prior belief of physicians is captured as labels on some of the patients. With this formulation, one goal is to learn a generalized Mahalanobis distance between patient xᵢ and patient xⱼ defined as:

dₘ(xᵢ,xⱼ)=\sqrt{(xᵢ−xⱼ)^{T}P(xᵢ−xⱼ)} (1)

where P ε [symbol] ^{N×N} is called the precision matrix. Matrix P is positive semi-definite and is used to incorporate the correlations between different feature dimensions. One aspect is to learn the optimal P such that the resulting distance metric has the following properties: 1) Within-class compactness: patients of the same label are close together; and 2) Between-class scatterness: patients of different labels are far away from each other. To formally measure these properties, we use two kinds of neighborhoods: 1) The homogeneous neighborhood of xᵢ, denoted as [symbol] ᵢ^{o}, is the k-nearest patients of xᵢ with the same label. 2) The heterogeneous neighborhood of xᵢ, denoted as [symbol] ᵢ^{e}, is the k-nearest patients of xᵢ with different labels.

Based on these two neighborhoods, we define the local compactness of point xᵢ as

$${C}_{i} = {\sum }_{{x}_{j} ∈ {𝒩}_{i}^{o}} {d}_{m}^{2} ( {x}_{i} , {x}_{j} ) \qquad ( 2 )$$

and the local scatterness of point xᵢ as

$${S}_{i} = {\sum }_{{x}_{k} ∈ {𝒩}_{i}^{e}} {d}_{m}^{2} ( {x}_{i} , {x}_{k} ) \qquad ( 3 )$$

The discriminability of the distance metric dₘ is defined as

$$𝒥 = \frac{{\sum }_{i} {C}_{i}}{{\sum }_{i} {S}_{i}} = \frac{{\sum }_{i} {\sum }_{{x}_{j} ∈ {𝒩}_{i}^{o}} {( {x}_{i} - {x}_{j} )}^{T} P ( {x}_{i} - {x}_{j} )}{{\sum }_{i} {\sum }_{{x}_{k} ∈ {𝒩}_{i}^{e}} {( {x}_{i} - {x}_{k} )}^{T} P ( {x}_{i} - {x}_{k} )} \qquad ( 4 )$$

The goal is to find a P that minimizes [symbol] , which is equivalent to minimizing the local compactness and maximizing the local scatterness simultaneously. In contrast with linear discriminant analysis, which seeks a discriminant subspace in a global sense, the localized supervised metric aims to learn a distance metric with enhanced local discriminability. To minimize [symbol] , we formulate the problem as a trace ratio minimization problem and use the decomposed Newton's method to find the solution.

Since P is a low-rank positive semi-definite matrix, we can decompose the precision matrix as P=WW^{T}, where W ε [symbol] ^{N×d} and d≦N. The distance metric can be rewritten as dₘ(xᵢ,x)=∥W^{T}xᵢ−W^{T}xⱼ∥. Therefore, the distance metric is equivalent to Euclidean distance over the low-dimensional projection W^{T}x.

Data Description and Feature Extraction: We have used the physiological data for 74 patients obtained from the MMIC II database in our experiments. Each patient is represented with 5 streams of sensor readings, sampled at 1 minute intervals: 1) Sp02, 2) heart rate (HR), 3) mean ABP (ABPmean), (4) systolic ABP (ABPSys), and diastolic ABP (ABPDias). All patients belong to one of two groups H or C. Those in group H (36 patients) had experienced Arterial Hypotensive Episode (AHE) events during the forecast window, whereas those in group C (38 patients) did not experience any AHE within the forecast window. The start of the forecast window is timestamped in the data set (T₀) and its duration is 1 hour, in which an episode of AHE can occur. For this study, we focus on a 2-hour window around T₀ for each patient. The data samples from two patients in H group show higher variability than those in C group. Physicians actually use the variability level of ABP to diagnose AHE.

We have used two different schemes to represent the 2-hour temporal data for each patient: a statistical time domain method and a wavelet domain method. In the former, we compute the mean and variance of data from each sensor for each patient. Thus, each patient is represented in the time domain with a 10-dimensional vector. In the latter, the wavelet coefficients of the 2-hour window from each sensor are computed. We use Daubechies-4 Wavelet and keep the top-10 coefficients. Finally, the coefficients from all 5 sensors are vectorized into a 50-dimensional feature vector for each patient.

From the feature extraction step described, we obtain 74 N-dimensional feature vectors where N=10 for the statistic method and N=50 for the Wavelet method. We then compare the following three distance metrics using the leave-one-out paradigm:

A) Expert uses Euclidean distance of the variance of the mean ABP; B) PCA uses Euclidean distance over low-dimensional points after PCA (an unsupervised metric learning algorithm); and C) LSML using the localized supervised metric learning method.

The performance metrics include k-NN classification error rate and precision@10 retrieval results. The precision@10 of a query point is computed by retrieving 10-nearest points with a specific distance metric and then computing the percentage of those retrieved points having the same label as the query point.

To have a fair comparison, both PCA and LSML project data into 1-dimensional space since the Expert method only uses one feature, i.e., the variance of mean ABP. Table 1 shows the classification results using 3-NN classifier, and Table 2 shows the retrieval results. As can be observed in both tables, LSML out-performs both expert and PCA on both statistical and Wavelet features, which confirms the importance of leveraging label information into the distance metric. We also observe that Wavelet features improve the performance significantly for LSML, where the classification error drops by half (from about 15% to less than 7%).

TABLE 1
Classification error comparison

| | Expert | PCA | LSML |
|---|---|---|---|
| Statistic features | 0.2295 | 0.2131 | 0.1475 |
| Wavelet features | NA | 0.2295 | 0.0656 |

TABLE 2
Precision@10 retrieval results

| | Expert | PCA | LSML |
|---|---|---|---|
| Statistic features | 0.6120 | 0.5355 | 0.6557 |
| Wavelet features | NA | 0.5410 | 0.7869 |

A method for deriving semantically sound similarity measures for retrieving patients represented by multi-dimensional time series has been described. The present methods use both statistical and wavelet based features to capture the characteristics of patients, and leverage localized supervised metric learning to incorporate physicians' expert domain knowledge.

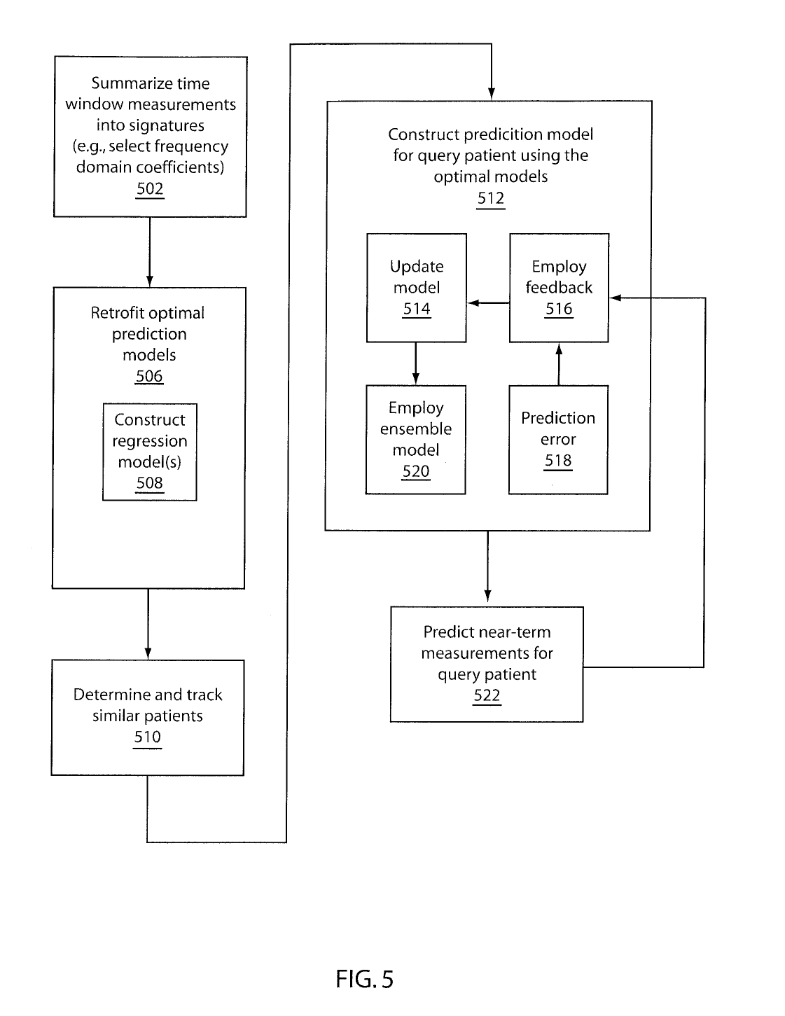

Referring to FIG. 5, a system/method for predicting near term measurements of a patient is illustratively shown in accordance with one exemplary embodiment. In block 502, time windows of patient measurements are summarized into signatures. This may be performed by converting raw data into the frequency domain (e.g., FFT, Wavelets, etc.) and selecting predetermined coefficients to comprise the signature. The frequency domain coefficients may represent time windows of a time series of measurements representing health status for a given patient. The time series of measurements may include one or more of heart rate, blood pressure, blood oxygen, electrocardiogram information, temperature, etc. The raw measurements are preferably summarized in real-time or near real-time (e.g., at the same time or within minutes or hours).

In block 506, optimal prediction models are retrofitted based on historical measurements. That is that we learn a regression model using historical data as input and current data as output, and update the model as more recent data becomes available. The retrofitting of optimal prediction models may include constructing a regression model using patient signatures to construct the optimal prediction models in block 508.

In block 510, similar patients and the signatures of the similar patients are determined and tracked for a query patient. The similar patients are determined by comparing signatures and computing a similarity score between a query patient and other patients. The other patients may include historical data or may include patients being monitored contemporaneously with the query patient. In block 512, a predictive model of the query patient is constructed using the optimal models from the similar patients. The predictive model can be updated by determining which of the optimal prediction models need to employed to predict near term measurements of the query patient based upon current conditions of the query patient in block 514. In block 516, feedback of a set of top similar patients is provided to enable updates to the optimal prediction models. In block 518, the feedback may include prediction error for updating the optimal prediction models.

The predictive model may be constructed by employing an ensemble model from the retrofitted optimal prediction models. The ensemble model may include weighted averages of all the retrofitted optimal prediction models where weights are proportional to a similarity between the query patient signature and the other patient signatures in block 520. In block 522, a near-term measurement for the query patient is predicted based on the predictive model. The prediction may be output as part of a patient report or provided wherever a physician, nurse or technician would find them useful. In one embodiment, models can be tested over time using the wealth of collected data.

Having described preferred embodiments of a system and method (which are intended to be illustrative and not limiting), it is noted that modifications and variations can be made by persons skilled in the art in light of the above teachings. It is therefore to be understood that changes may be made in the particular embodiments disclosed which are within the scope of the invention as outlined by the appended claims. Having thus described aspects of the invention, with the details and particularity required by the patent laws, what is claimed and desired protected by Letters Patent is set forth in the appended claims.

What is claimed is:

1. A system to predict near term measurements of a patient, comprising:
a stream processor configured to summarize raw measurements from patients into signatures and construct optimal prediction models using previously obtained signatures;
a similar patient tracker configured to monitor similar patient information relative to a query patient, the similar patient information being determined based on a similarity between the query patient and the signatures of other patients; and
a model analyzer configured to employ retrofitted optimal prediction models from similar patients to predict near term measurements of the query patient.

2. The system as recited in claim 1, wherein the stream processor summarizes raw measurements in real-time or near real-time.

3. The system as recited in claim 1, wherein the signatures include frequency domain coefficients which represent time windows of a time series of measurements representing health status for a given patient.

4. The system as recited in claim 3, wherein the time series of measurements includes one or more of heart rate, blood pressure, blood oxygen, electrocardiogram information and temperature.

5. The system as recited in claim 1, wherein the similar patient tracker updates which optimal prediction models that are employed to predict near term measurements of the query patient based upon current conditions of the query patient.

6. The system as recited in claim 5, wherein the model analyzer determines a set of top similar patients and feeds back this information to enable the similar patient tracker to update the optimal prediction models.

7. The system as recited in claim 6, wherein the model analyzer employs prediction error as feedback for updating the optimal prediction models.

8. The system as recited in claim 1, wherein the stream processor constructs a regression model using patient signatures to construct the optimal prediction models.

9. The system as recited in claim 1, wherein the model analyzer employs an ensemble model from the retrofitted optimal prediction models to create the prediction model.

10. The system as recited in claim 1, wherein the ensemble model includes weighted averages of all the retrofitted optimal prediction models where weights are proportional to a similarity between the query patient signature and the other patient signatures.

11. A method for predicting near term measurements of a patient, comprising:
summarizing time windows of patient measurements into signatures;
retrofitting optimal prediction models based on historical measurements;
tracking similar patients and signatures of the similar patients for a query patient;
constructing a predictive model of the query patient using the optimal models from the similar patients; and
predicting a near-term measurement for the query patient based on the predictive model.

12. The method as recited in claim 11, wherein summarizing time windows includes summarizing raw measurements in real-time or near real-time.

13. The method as recited in claim 11, wherein the signatures include frequency domain coefficients which represent the time windows of a time series of measurements representing health status for a given patient.

14. The method as recited in claim 13, wherein the time series of measurements includes one or more of heart rate, blood pressure, blood oxygen, electrocardiogram information and temperature.

15. The method as recited in claim 11, wherein constructing includes updating which of the optimal prediction models are employed to predict near term measurements of the query patient based upon current conditions of the query patient.

16. The method as recited in claim 15, further comprising providing feedback of a set of top similar patients to enable updates to the optimal prediction models.

17. The method as recited in claim 16, wherein the feedback includes prediction error for updating the optimal prediction models.

18. The method as recited in claim 11, wherein retrofitting optimal prediction models includes constructing a regression model using patient signatures to construct the optimal prediction models.

19. The method as recited in claim 11, wherein constructing a predictive model includes employing an ensemble model from the retrofitted optimal prediction models to create the prediction model.

20. The method as recited in claim 19, wherein the ensemble model includes weighted averages of all the retrofitted optimal prediction models where weights are proportional to a similarity between the query patient signature and the other patient signatures.

21. A computer readable storage medium comprising a computer readable program for predicting near term measurements of a patient, wherein the computer readable program when executed on a computer causes the computer to perform the steps of:
summarizing time windows of patient measurements into signatures;
retrofitting optimal prediction models based on historical measurements;
tracking similar patients and signatures of the similar patients for a query patient;
constructing a predictive model of the query patient using the optimal models from the similar patients; and
predicting a near-term measurement for the query patient based on the predictive model.

22. The computer readable storage as recited in claim 21, wherein summarizing time windows includes summarizing raw measurements in real-time or near real-time,

23. The computer readable storage as recited in claim 21, wherein the signatures include frequency domain coefficients which represent the time windows of a time series of measurements representing health status for a given patient.

24. The computer readable storage as recited in claim 23, wherein the time series of measurements includes one or more of heart rate, blood pressure, blood oxygen, electrocardiogram information and temperature.

25. The computer readable storage as recited in claim 21, wherein constructing a predictive model includes employing an ensemble model from the retrofitted optimal prediction models to create the prediction model, wherein the ensemble model includes weighted averages of all the retrofitted optimal prediction models where weights are proportional to a similarity between the query patient signature and the other patient signatures.
